(19) United States
(12) Patent Application Publication
(10) Pub. No.: US 2022/0031874 A1
(43) Pub. Date: Feb. 3, 2022

(54) SHOPPING CART SANITIZATION DEVICE

(71) Applicants: Nazih Ibrahim, Boca Raton, FL (US); Sami Ibrahim, Boca Raton, FL (US); Errin Gnadinger, Boca Raton, FL (US)

(72) Inventors: Nazih Ibrahim, Boca Raton, FL (US); Sami Ibrahim, Boca Raton, FL (US); Errin Gnadinger, Boca Raton, FL (US)

(21) Appl. No.: 16/945,681

(22) Filed: Jul. 31, 2020

(51) Int. Cl. A61L 2/10; A61L 2/26

(52) U.S. Cl. CPC A61L 2/10; A61L 2202/14; A61L 2202/11; A61L 2/26

(57) ABSTRACT

A shopping cart sanitization device, including a main body to receive at least one shopping cart therein, and a plurality of sanitization units, each of the plurality of sanitization units including a top illumination unit disposed on at least a portion of a top of the main body to illuminate a first UV light on the at least one shopping cart from above, such that the top illumination unit is oriented toward a ground surface, a first side illumination unit disposed on at least a portion of a first side of the main body to illuminate a second UV light on a first side of the at least one shopping cart, and a second side illumination unit disposed on at least a portion of a second side of the main body opposite with respect to the first side to illuminate a third UV light on a second side of the at least one shopping cart.

### BACKGROUND

### 1. Field

The present general inventive concept relates generally to a sanitization device, and particularly, to a shopping cart sanitization device.

### 2. Description of the Related Art

A shopping cart is a wheeled cart that is usually provided by a store, such as a supermarket that allows a customer to store merchandise therein. The shopping cart improves a shopping experience of the customer by providing the customer a means to collect all the merchandise in a convenient location prior to purchasing the merchandise.

Unsurprisingly, the shopping cart is used by multiple customers throughout each day. As such, the shopping cart is touched by each customer, which can quickly lead to the spread of a pathogen, such as bacteria, viruses, fungi, and/or a parasite. For example, the recent pandemic involving the severe acute respiratory syndrome coronavirus 2 (SARS-CoV-2) is affecting many people worldwide. A known vector of transmission between humans of the SARS-CoV-2 is touching a contaminated surface and then touching a face.

Some methods of cleaning the shopping cart include spraying a handle with a disinfectant and/or wiping the handle of the shopping cart with a disinfectant wipe. However, these methods require active participation by the customer and/or an employee of the store to either spray and/or wipe the shopping cart. Also, other portions of the shopping cart are prone to be touched which can keep pathogens present.

Therefore, there is a need for a shopping cart sanitization device that can quickly disinfect a line of shopping carts.

### SUMMARY

The present general inventive concept provides a shopping cart sanitization device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a shopping cart sanitization device, including a main body to receive at least one shopping cart therein, and a plurality of sanitization units, each of the plurality of sanitization units including a top illumination unit disposed on at least a portion of a top of the main body to illuminate a first UV light on the at least one shopping cart from above, such that the top illumination unit is oriented toward a ground surface, a first side illumination unit disposed on at least a portion of a first side of the main body to illuminate a second UV light on a first side of the at least one shopping cart, and a second side illumination unit disposed on at least a portion of a second side of the main body opposite with respect to the first side to illuminate a third UV light on a second side of the at least one shopping cart.

The main body may include a barrier disposed on at least one portion of the main body, and an inclined surface disposed within at least at least one portion of the main body to facilitate extraction of the at least one shopping cart from the main body.

The top illumination unit, the first side illumination unit, and the second side illumination unit each illuminate in response to the barrier being sufficiently closed.

The top illumination unit, the first side illumination unit, and the second side illumination unit each may include at least one of any form of UV light.

The main body may allow visible light with respect to humans to move therethrough and prevent the UV light from moving therethrough.

The main body may prevent the UV light from moving therethrough using polycarbonate or other transparent or translucent materials, either by merits of the other transparent or translucent materials or an addition of specialty coatings.

The main body may allow visible light with respect to humans to move therethrough and reflect or absorb the UV light toward the interior of the main body.

The main body may reflect or absorb the UV light using barium sulphate or any other reflective material.

At least one of the first side illumination unit and the second side illumination unit may oscillate in a first direction or a second direction for a predetermined number of times.

The shopping cart sanitization device may further include a barrier sanitization unit disposed on at least a portion of the main body to automatically illuminate at least one barrier UV light on the at least one shopping cart in response to the main body receiving the at least one shopping cart.

The shopping cart sanitization device may further include a sensor disposed on at least a portion of the main body to detect the at least one shopping cart within the main body.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a rear perspective view of a shopping cart sanitization device, according to an exemplary embodiment of the present general inventive concept;

FIG. 2 illustrates a rear view of the shopping cart sanitization device, according to an exemplary embodiment of the present general inventive concept;

FIG. 3A illustrates one of a plurality of sanitization units oriented in a first position, according to another exemplary embodiment of the present general inventive concept; and

FIG. 3B illustrates one of the plurality of sanitization units oriented in a second position, according to another exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Shopping Cart Sanitization Device 100
Main Body 110
Body Sections 111
Barrier 112
Barrier-cleaning apparatuses 113
Inclined Surface 114
Inner Surface 115
Sanitization Units 120
Top Illumination Unit 121
First Side Illumination Unit 122
Second Side Illumination Unit 123
Barrier Sanitization Unit 130
First Barrier Illumination Unit 131
Second Barrier Illumination Unit 132
Rail Assembly 140
Rails 141
Cart Lock 142
Sensor 150
Power Source 160
Sanitization Units 220
First Side Illumination Unit 221
Second Side Illumination Unit 222

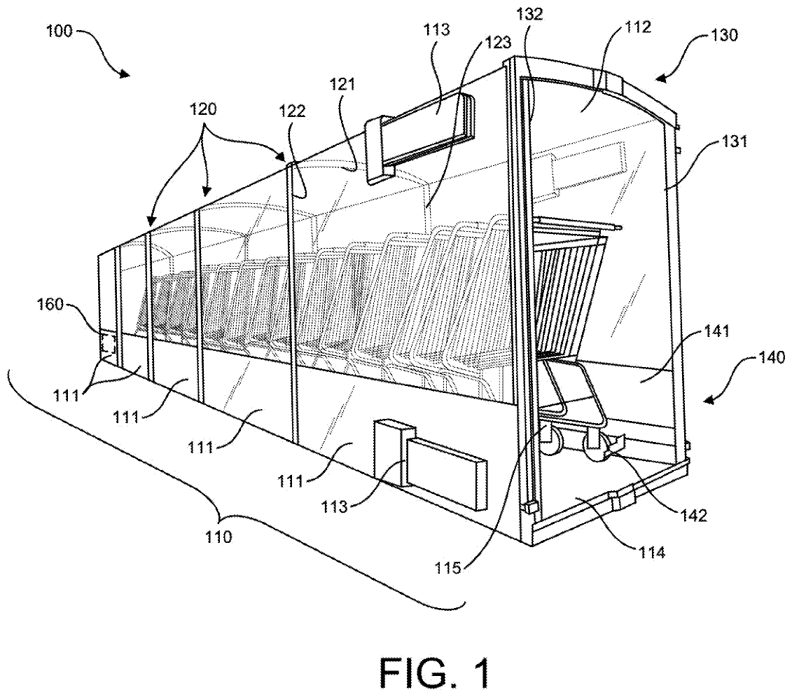

FIG. 1 illustrates a rear perspective view of a shopping cart sanitization device 100, according to an exemplary embodiment of the present general inventive concept.

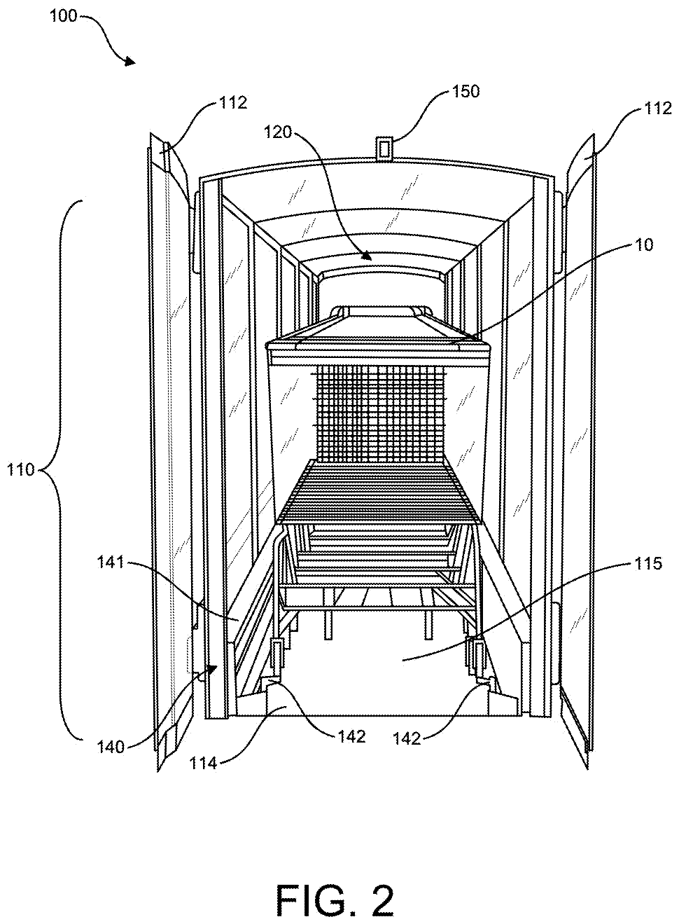

FIG. 2 illustrates a rear view of the shopping cart sanitization device 100, according to an exemplary embodiment of the present general inventive concept.

The shopping cart sanitization device 100 may be constructed from at least one of glass, metal, plastic, wood, and rubber, etc., but is not limited thereto and may be constructed from any material.

The shopping cart sanitization device 100 may include a main body 110, a plurality of sanitization units 120, a barrier sanitization unit 130, a rail assembly 140, a sensor 150, and a power source 160, but is not limited thereto.

Referring to FIGS. 1 and 2, the main body 110 may be constructed of an ultraviolet (UV) light filtered glass, such as polycarbonate or other transparent or translucent materials, either by merits of the other transparent or translucent materials or an addition of specialty coatings. In particular, the main body 110 may allow visible light with respect to humans to move therethrough, but prevent UV light from moving (i.e. transmission) therethrough. For example, UV light is electromagnetic radiation having a wavelength range of between 400 nanonmeters (nm) and 100 nm. Furthermore, the UV light may include UVA, UVB, UVC, near ultraviolet, middle ultraviolet, far ultraviolet, vacuum ultraviolet, and any other form of ultraviolet light. Also, instead of UV filtered glass, the main body 110 may have a UV coating and/or tinting that prevents UV light from moving therethrough, such as organic dyes and/or metallic oxide pigments. The main body 110 may be constructed to be highly durable and shatterproof.

Referring again to FIGS. 1 and 2, the main body 110 is illustrated to have a rectangular prism shape. However, the main body 110 may be rectangular, circular, conical, triangular, pentagonal, hexagonal, heptagonal, octagonal, or any other shape known to one of ordinary skill in the art, but is not limited thereto.

Alternatively, the main body 110 may use a UV reflective coating, such that the UV light within the main body 110 may reflect toward an interior of the main body 110. For example, the UV reflective coating may include barium sulphate or any other reflective material.

The main body 110 may include a plurality of body sections 111, a barrier 112, a plurality of barrier-cleaning apparatuses 113, an inclined surface 114, and an inner surface 115, but is not limited thereto.

Each of the plurality of body sections 111 may have a length and/or a width equivalent to each other. In other words, each of the plurality of body sections 111 may be equivalent in size. However, the plurality of body sections 111 may differ in length and/or width based on a preference of a user and/or a manufacturer.

Each of the plurality of body sections 111 may be removably connected to each other, such that the main body 110 may vary in length. As such, the main body 110 is formed from the plurality of body sections 111.

Referring again to FIGS. 1A through 10, the barrier 112 may be disposed on at least a portion of a first end of the main body 110. The barrier 112 may include two barriers or a single barrier. The barrier 112 may be constructed of the UV light filtered glass, similar to the main body 110. In other words, the barrier 112 may prevent the UV light from moving therethrough and/or reflecting the UV light toward the interior of the main body 112.

The barrier 112 may open allow at least one shopping cart 10 to be received through an entrance into the main body 110, such that the main body 110 may store the at least one shopping cart 10 therein.

Although the present general concept is directed to a shopping cart sanitization device 100 that allows at least one shopping cart 10 to be stored therein, the present general inventive concept is not limited thereto. In other words, the shopping cart 10 may be a shopping cart, a wheelchair, a gurney, a motorized device, a scooter, or any other type of object that may require sanitization. This applied to all embodiments of the present general inventive concept.

The plurality of barrier-cleaning apparatuses 113 may be disposed on at least one portion of the main body 110, such that a first set of the plurality of barrier-cleaning apparatuses 113 is disposed on a first side of the main body 110 and a second set of the plurality of barrier-cleaning apparatuses 113 disposed on a second side of the main body 110. The plurality of barrier-cleaning apparatuses 113 may be removably connected to the barrier 112. Moreover, the barrier 112 may move in a first lateral direction or a second lateral direction within the barrier-cleaning apparatuses 113 from extended in a first position to at least partially retracted in a second position while the barrier 112 is opened. As such, the barrier-cleaning apparatuses 113 may receive the barrier 112 along an outer surface of the main body 110. Therefore, the barrier-cleaning apparatuses 113 may prevent damage to the barrier 112 while the barrier 112 is disposed on the first side and the second side the main body 110.

Conversely, the barrier 112 may move in the second lateral direction or the first lateral direction within the barrier-cleaning apparatuses 113 from retracted in the second position to extended in the first position, such that the barrier 112 may be closed.

The inclined surface 114 may be disposed within at least one portion of the main body 110. The inclined surface 114 may facilitate entry of the at least one shopping cart 10 therein. Alternatively, the inclined surface 114 may facilitate extraction of the at least one shopping cart 10 from within the main body 110, such that the inclined surface 114 may allow the at least one shopping cart 10 to roll down thereupon.

The inner surface 115 may be disposed within the interior of the main body 110. The inner surface 115 may receive the at least one shopping cart 10 thereupon. Additionally, the inner surface 115 may be a conveyor belt to move the at least one shopping cart 10 into the interior of the main body 110 and/or an exterior (i.e. outside) of the main body 110.

Each of the plurality of sanitization units 120 may include a top illumination unit 121, a first side illumination unit 122, and a second side illumination unit 123, but is not limited thereto.

The plurality of sanitization units 120 may be removably disposed on at least a portion of each connection between each of the plurality of body sections 111.

The top illumination unit 121 may include a first UV light, but is not limited thereto.

The first side illumination unit 122 may include a second UV light, but is not limited thereto.

The second side illumination unit 123 may include a third UV light, but is not limited thereto. More specifically, the first UV light, the second UV light, and/or the third UV light (hereinafter, will be referred to as the UV lights) used may be a UVC light, such that the UV lights is a germicidal to illuminate, irradiate, and/or eliminate a pathogen.

The top illumination unit 121 may be disposed on at least a portion of a top of the main body 110, such that the top illumination unit 121 is oriented toward the ground surface. The first side illumination unit 122 may be disposed on at least a portion of the first side of the main body 110. The second side illumination unit 123 may be disposed on at least a portion of the second side of the main body 110. Also, the top illumination unit 121 may be perpendicularly disposed away from the first side illumination unit 122 with respect to a first direction, and/or away from the second side illumination unit 123 with respect to a second direction opposite to the first direction. Alternatively, the top illumination unit 121 may be arcuately disposed away from the first side illumination unit 122 with respect to the first direction, and/or away from the second side illumination unit 123 with respect to the second direction opposite to the first direction.

The top illumination unit 121 may illuminate the at least one shopping cart 10 from above in response to the barrier 112 being sufficiently closed. The first side illumination unit 122 and/or the second side illumination unit 123 may illuminate a first side and/or a second side of the at least one shopping cart 10 in response to the barrier 112 being sufficiently closed. In other words, the top illumination unit 121, the first side illumination unit 122, and/or the second side illumination unit 123 may illuminate automatically after the barrier 112 is closed.

Therefore, the top illumination unit 121, the first side illumination unit 122, and/or the second side illumination unit 123 may sanitize the at least one shopping cart 10 in response to illuminating the UV lights thereupon.

Furthermore, the top illumination unit 121, the first side illumination unit 122, and/or the second side illumination unit 123 may stop illuminating in response to opening the barrier 112. As such, the UV lights emitted from the top illumination unit 121, the first side illumination unit 122, and/or the second side illumination unit 123 may be prevented from causing injury to a living organism outside the main body 110.

The barrier sanitization unit 130 may include a first barrier illumination unit 131 and a second barrier illumination unit 132, but is not limited thereto.

The barrier sanitization unit 130 may be disposed within at least one portion of the main body 110, such as a barrier frame of the barrier 112.

The first barrier illumination unit 131 may include a first barrier UV light, but is not limited thereto. The second barrier illumination unit 132 may include a second barrier UV light, but is not limited thereto. Collectively, the first barrier UV light and the second barrier UV lights may be referred to as at least one barrier UV light. More specifically, the at least one barrier UV light used may be a UVC light, such that the at least one barrier UV light is a germicidal to illuminate, irradiate, and/or eliminate a pathogen.

The first barrier illumination unit 131 may be disposed on at least a portion of the first side of the main body 110. The second barrier illumination unit 132 may be disposed on at least a portion of the second side of the main body 110.

The first barrier illumination unit 131 and/or the second barrier illumination unit 132 may illuminate in response to the barrier 112 being sufficiently closed. In other words, the first barrier illumination unit 131 and/or the second barrier illumination unit 132 may illuminate automatically after the barrier 112 is closed.

Therefore, the first barrier illumination unit 131 and/or the second barrier illumination unit 132 may sanitize the at least one shopping cart 10 in response to illuminating the at least one barrier UV light thereupon.

Furthermore, the first side illumination unit 131 and/or the second side illumination unit 132 may stop illuminating in response to opening the barrier 112. As such, the at least one barrier UV light emitted from the first side illumination unit 131 and/or the second side illumination unit 132 may be prevented from causing injury to a living organism outside the main body 110.

The rail assembly 140 may include a plurality of rails 141 and a cart lock 142, but is not limited thereto.

The plurality of rails 141 may be disposed on at least a portion of a base within the main body 110. The plurality of rails 141 may receive and connect to the at least one shopping cart 10 therein. Moreover, the plurality of rails 141 may prevent the at least one shopping cart 10 from lateral movement within the main body 110, such that the at least one shopping cart 10 may move in a straight line within the main body 110.

Furthermore, the plurality of rails 141 may move the at least one shopping cart 10 therein in response to the at least one shopping cart 10 contacting the plurality of rails 141. In other words, the plurality of rails 141 may automatically move the at least one shopping cart 10 from the first end of the main body 110 to at least partially toward the second end of the main body 110 in response to a number of shopping carts already within the main body 110 and/or being moved into the main body 110.

The cart lock 142 may be disposed within at least a portion of the rails 141. The cart lock 142 may prevent at least one shopping cart 10 from moving out of the plurality of rails 141. However, the cart lock 142 may release the at least one shopping cart 10 in response to receive a predetermined amount of force thereto. In other words, the cart lock 142 may release the at least one shopping cart 10 in response to retrieval by the user, but would not release the at least one shopping cart 10 while remaining within the plurality of rails 141.

The sensor 150 may be disposed on at least a portion of the main body 110. The sensor 150 may detect approach of the at least one shopping cart 10 toward the barrier 112 and/or the main body 110. Subsequently, the barrier 112 may open in response to detection of the at least one shopping cart 10 by the sensor 150, or alternatively, detection of a presence of human or non-human beings, which would also preclude the UV system. Moreover, the plurality of sanitization units 120, the barrier sanitization unit 130, and/or the rail assembly 140 may activate in response to detection of the at least one shopping cart 10 being received within the main body 110 and/or closure of the barrier 112. Also, the barrier 112 may close in response to detection of the at least one shopping cart 10 moved within the main body 110.

After plurality of auxiliary sanitization units 120 and/or the barrier sanitization unit 130 have completed sanitization, the barrier 112 may open in response to detection of completion by the sensor 150.

Alternatively, the sensor 150 may be replaced with a button and/or a control panel, such that manual input from the user controls operation of the barrier 112, the plurality of sanitization units 120, the barrier sanitization unit 130, and/or the rail assembly 140.

The power source 160 may include a battery, a solar cell, and a power inlet, but is not limited thereto.

The power source may provide power to the plurality of sanitization units 120, the barrier sanitization unit 130, the rail assembly 140, and/or the sensor 150.

Therefore, the shopping cart sanitization device 100 may disinfect a plurality of shopping carts 10 due to the numerous UV illumination units. Also, the shopping cart sanitization device 100 may eliminate all pathogens from the at least one shopping cart 10 due to the plurality of sanitization units 120.

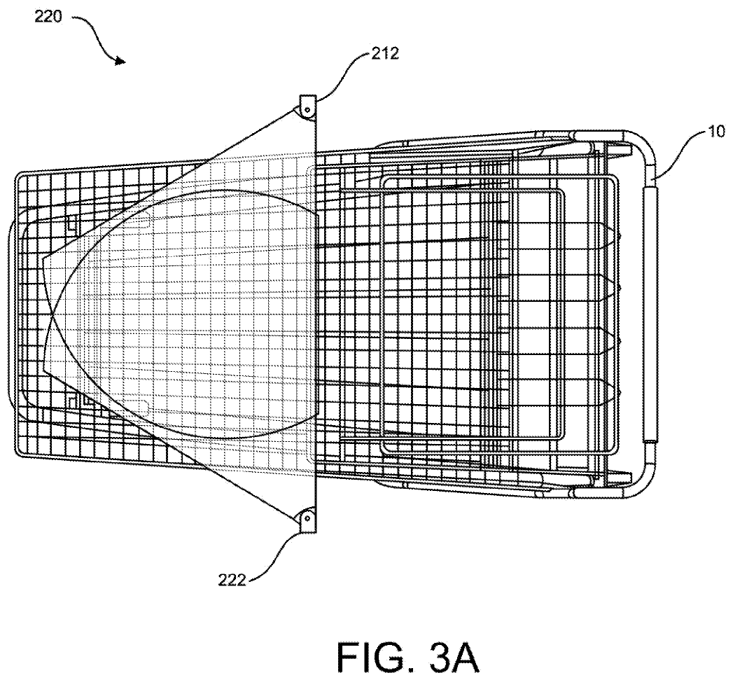

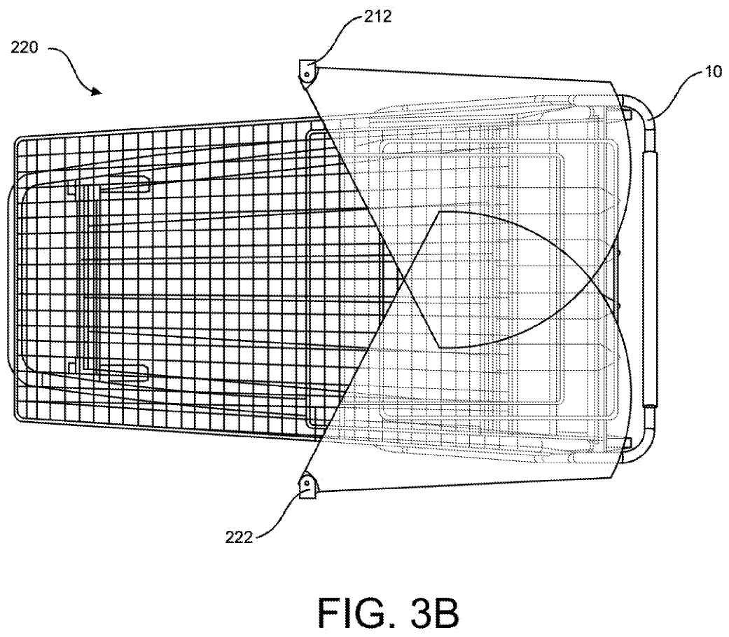

FIG. 3A illustrates one of a plurality of sanitization units 220 oriented in a first position, according to another exemplary embodiment of the present general inventive concept.

FIG. 3B illustrates one of the plurality of sanitization units 220 oriented in a second position, according to another exemplary embodiment of the present general inventive concept.

Each of the plurality of sanitization units 220 may include a first side illumination unit 221 and a second side illumination unit 222, but is not limited thereto.

The plurality of sanitization units 220 may be removably disposed on at least a portion of each connection between each of the plurality of body sections 111. Also, the plurality of sanitization units 220 may be used on the main body 110 instead of and/or in addition to the plurality of sanitization units 120, as described above.

The first side illumination unit 221 and the second side illumination unit 222 may include a UV light, but is not limited thereto. More specifically, the UV light used may be a UVC light, such that the UV light is a germicidal to illuminate, irradiate, and/or eliminate a pathogen.

The first side illumination unit 221 may be disposed on at least a portion of the first side of the main body 110. The second side illumination unit 222 may be disposed on at least a portion of the second side of the main body 110.

The first side illumination unit 221 and/or the second side illumination unit 222 may illuminate in response to closure of the barrier 112. In other words, the first side illumination unit 221 and/or the second side illumination unit 222 may illuminate automatically after the barrier 112 is closed.

Referring to FIGS. 3A and 3B, additionally, the first side illumination unit 221 and/or the second side illumination unit 222 may include a motor therein to oscillate in a first direction or a second direction from the first end of the main body 110 in a first position to at least partially toward the second end of the main body 110 in a second position, such that the first side illumination unit 221 and/or the second side illumination unit 222 may move the UV light over a greater surface area of the at least one shopping cart 10. Additionally, the first side illumination unit 221 and/or the second side illumination unit 222 may oscillate in the second direction or the first direction from the second end of the main body 110 in the second position to the first end of the main body 110 in the first position after reaching the second position. In other words, the first side illumination unit 221 and/or the second side illumination unit 222 may repeatedly move back and forth between the first position and/or the second position for a predetermined number of times. For example, the first side illumination unit 221 and/or the second side illumination unit 222 may oscillate once, twice, or three times based on the preference of the user and/or the manufacturer.

Also, the first side illumination unit 221 and/or the second side illumination unit 222 may oscillate simultaneously in similar directions and/or independently in different directions with respect to each other.

Therefore, the first side illumination unit 221 and/or the second side illumination unit 222 may sanitize the at least one shopping cart 10 in response to illuminating the UV light thereupon.

Furthermore, the first side illumination unit 221 and/or the second side illumination unit 222 may stop illuminating in response to opening the barrier 112. As such, the UV light emitted from the first side illumination unit 221 and/or the second side illumination unit 222 may be prevented from causing injury to a living organism outside the main body 110.

The present general inventive concept may include a shopping cart sanitization device 100, including a main body 110 to receive at least one shopping cart 10 therein, and a plurality of sanitization units 120, each of the plurality of sanitization units 120 including a top illumination unit 121 disposed on at least a portion of a top of the main body 110 to illuminate a first UV light on the at least one shopping cart 10 from above, such that the top illumination unit 121 is oriented toward a ground surface, a first side illumination unit 122 disposed on at least a portion of a first side of the main body 110 to illuminate a second UV light on a first side of the at least one shopping cart 10, and a second side illumination unit 123 disposed on at least a portion of a second side of the main body 110 opposite with respect to the first side to illuminate a third UV light on a second side of the at least one shopping cart 10.

The main body 110 may include a barrier 112 disposed on at least one portion of the main body 110, and an inclined surface 114 disposed within at least one portion of the main body 110 to facilitate extraction of the at least one shopping cart 10 from the main body 110.

The top illumination unit 121, the first side illumination unit 122, and the second side illumination unit 123 each illuminate in response to the barrier 112 being sufficiently closed.

The top illumination unit 121, the first side illumination unit 122, and the second side illumination unit 123 each may include at least one of any type of UV light.

The main body 110 may allow visible light with respect to humans to move therethrough and prevent the UV light from moving therethrough.

The main body 110 may prevent the UV light from moving therethrough using polycarbonate or other transparent or translucent materials, either by merits of the other transparent or translucent materials or an addition of specialty coatings.

The main body 110 may allow visible light with respect to humans to move therethrough and reflect or absorb the UV light toward the interior of the main body 110.

The main body 110 may reflect or absorb the UV light using barium sulphate or any other reflective material.

At least one of the first side illumination unit 221 and the second side illumination unit 222 may oscillate in a first direction or a second direction for a predetermined number of times.

The shopping cart sanitization device 100 may further include a barrier sanitization unit 130 disposed on at least a portion of the main body 110 to automatically illuminate at least one barrier UV light on the at least one shopping cart 10 in response to the main body 110 receiving the at least one shopping cart 10.

The shopping cart sanitization device 100 may further include a sensor 150 disposed on at least a portion of the main body 110 to detect the at least one shopping cart 10 within the main body 110.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A shopping cart sanitization device, comprising:
a main body to receive at least one shopping cart therein; and
a plurality of sanitization units, each of the plurality of sanitization units comprising:
a top illumination unit disposed on at least a portion of a top of the main body to illuminate a first UV light on the at least one shopping cart from above, such that the top illumination unit is oriented toward a ground surface,
a first side illumination unit disposed on at least a portion of a first side of the main body to illuminate a second UV light on a first side of the at least one shopping cart, and
a second side illumination unit disposed on at least a portion of a second side of the main body opposite with respect to the first side to illuminate a third UV light on a second side of the at least one shopping cart.

2. The shopping cart sanitization device of claim 1, wherein the main body comprises:
a barrier disposed on at least one portion of the main body; and
an inclined surface disposed within at least at least one portion of the main body to facilitate extraction of the at least one shopping cart from the main body.

3. The shopping cart sanitization device of claim 2, wherein the top illumination unit, the first side illumination unit, and the second side illumination unit each illuminate in response to the barrier being sufficiently closed.

4. The shopping cart sanitization device of claim 1, wherein the top illumination unit, the first side illumination unit, and the second side illumination unit each comprise at least one of any type of UV light.

5. The shopping cart sanitization device of claim 1, wherein the main body allows visible light with respect to humans to move therethrough and prevent the UV light from moving therethrough.

6. The shopping cart sanitization device of claim 5, wherein the main body prevents the UV light from moving therethrough using polycarbonate or other transparent or translucent materials, either by merits of the other transparent or translucent materials or an addition of specialty coatings.

7. The shopping cart sanitization device of claim 1, wherein the main body allows visible light with respect to humans to move therethrough and reflect or absorb the UV light toward the interior of the main body.

8. The shopping cart sanitization device of claim 7, wherein the main body reflects or absorbs the UV light using barium sulphate or any other reflective material.

9. The shopping cart sanitization device of claim 1, wherein at least one of the first side illumination unit and the second side illumination unit oscillate in a first direction or a second direction for a predetermined number of times.

10. The shopping cart sanitization device of claim 1, further comprising:
a barrier sanitization unit disposed on at least a portion of the main body to automatically illuminate at least one barrier UV light on the at least one shopping cart in response to the main body receiving the at least one shopping cart.

11. The shopping cart sanitization device of claim 1, further comprising:
a sensor disposed on at least a portion of the main body to detect the at least one shopping cart within the main body.
